(19) United States
(12) Patent Application Publication
(10) Pub. No.: US 2025/0367231 A1
(43) Pub. Date: Dec. 4, 2025

(54) MULTIFUNCTIONAL BIOADHESIVE DESIGNED WITH CHEMICAL FUNCTIONALITY INDUCED SUTURED BIOLOGICAL BUILDING BLOCKS

(71) Applicant: The Regents of the University of California, Oakland, CA (US)

(72) Inventors: Nasim Annabi, Los Angeles, CA (US); Avijit Baidya, Los Angeles, CA (US)

(73) Assignee: The Regents of the University of California, Oakland, CA (US)

(21) Appl. No.: 18/868,628

(22) PCT Filed: Jun. 15, 2023
(86) PCT No.: PCT/US2023/068524

§ 371 Date: Nov. 22, 2024

Related U.S. Application Data
(60) Provisional application No. 63/353,149, filed on Jun. 17, 2022.

(51) Int. Cl. A61K 31/785; A61K 9/06; A61K 47/42

(52) U.S. Cl. CPC A61K 31/785; A61K 9/06; A61K 47/42

(57) ABSTRACT

A neurotransmitter moiety, dopamine, was chemically modified with a nitro-group to explore its influence on synthesizing a multifunctional biomaterial for therapeutic applications. Chemically, by manipulating the aromatic-electron density. while nitro-group prevented the self-oxidation of catecholic domain. this facilitated the aromatic suturing of nitrocatechol moieties, forming a novel macromolecular structure. Incorporation of the sutured-nitrocatecholic moieties in a gelatin-based hydrogel introduced extended pi-electron delocalization mediated electroconductive microenvironment and maintained its adhesive properties originated from the catecholic domains, forming a multi-functional bioadhesive for tissue repairing. Embodiments of the invention include engineered hydrogels enriched with multi-mode noncovalent interactions and excellent mechano-physical properties that are useful for diverse biomedical applications.

### CROSS REFERENCE TO RELATED APPLICATIONS

This application claims the benefit under 35 U.S.C. Section 119 (c) of co-pending and commonly-assigned U.S. Provisional Patent Application No. 63/353,149, filed Jun. 17, 2022, entitled “A MULTIFUNCTIONAL BIOADHESIVE DESIGNED WITH CHEMICAL FUNCTIONALITY INDUCED SUTURED BIOLOGICAL BUILDING BLOCKS”, which application is incorporated by reference herein.

### STATEMENT REGARDING FEDERALLY SPONSORED RESEARCH AND DEVELOPMENT

This invention was made with goverment support under HL140618 and EB023052, awarded by the National Institutes of Health. The government has certain rights in the invention.

### TECHNICAL FIELD

The invention relates to methods of synthesizing macromolecules by tuning the electron density of biologically relevant small biomolecules and developing multifunctional biomaterials for biomedical applications.

### BACKGROUND OF THE INVENTION

Chemical structure of molecules play an important role defining their spatial interactions with biological macromolecules like enzymes, proteins, and others in cellular pathways^{[1]}. Simultaneously, chemical functionalities presented in the biomolecular skeletons also contribute equally to control the reactivity of cellular chemical reactions^{[1c, 2]}. Thus, along with the structural aspect of the bioactive molecules, their chemical functionalities regulate the cellular physio-chemical microenvironments, allowing healthy tissue function^{[3]}. For instance, in neural system, catecholamines, a group of neurotransmitter molecules with catecholic structures, guide the biological signaling mechanisms and support nerve function^{[1c, 4]}. Despite having structural similarity. functional diversity in these neurotransmitter molecules is essential and enables sequential cellular responses. Deficiency in any of these bioactive molecules could result sever neural disorders and complex diseases^{[4-5]}.

Meanwhile, the role of chemical functionalities in biomolecules and their therapeutic effects are predominantly observed during the synthesis of the drug molecules^{[2, 6]}. Particularly for aromatic biomolecules, chemical substitutions either increase the electron density in aromatic moieties or deactivate the ring with electron withdrawing capacity^{[7]}. Thus, these chemical substituents alter the parent molecular reactivity and regulate the specific biological interactions. Nitro (—NO₂) is one of such biologically active, chemical functionalities with a large deficit of negative charge, charge which can modulate the electron density of aromatic biomolecules^{[8]}. Therefore, it has become an essential part of designing drug molecules, facilitating the enzymatic activities in cellular environment^{[9]}.

Recently, several nitro-based molecules are utilized for the treatment of cardiovascular diseases, anxiety disorders, Parkinson's disease, and even as anticancer agent^{[8-9]}. In addition, nitrocatecholic moieties also contribute in neural signaling pathways^{[10]}. While nitro-group chemically interacts with intercellular enzymes to release nitric oxide, its specific position in the drug molecules' skeleton enhances the therapeutic actions^{[8]}. Particularly for Parkinson's disease, nitro-group acts as a catechol-O-methyltransferase (COMT) inhibitor and slows down levodopa metabolism, balancing the dopamine concentration in the nerval system^{[8]}. However, the effect of nitro-group on aromatic biomolecules to design new macromolecules and developing biologically relevant, multifunctional biomaterials with tunable macroscopic physical properties for medical applications has not been explored.

There is a need in the art for new materials and methods useful for making and/or using new biomaterials with tunable macroscopic physical properties for medical applications. The present invention addresses this unmet need.

### SUMMARY OF THE INVENTION

As disclosed herein. by manipulating the electron density of a naturally derived neurotransmitter molecule^{[4]}, dopamine (Dopa), with nitro-functionality, we designed a new macromolecular structure that can introduce biologically relevant macroscopic multifunctionality for diverse biomedical applications. As discussed below, the addition of nitro groups on to Dopa can, for example, facilitate the chemical suturing of aromatic domains of nitrodopamine molecules (nDopa). Moreover, the introduction of methacrylated groups to nDopa can provide a polymeric backbone to generate sutured-nitrocatacholic strands, a composition termed herein as “S-nCAT”. In such compositions, chemically, strong electron withdrawing nitro group easily perturbed the aromatic electron density of nitrocatecholic domain and enabled the suturing of nitrocatechol moieties to regain its aromaticity through radical transfer mechanisms.

In order to examine the uses of S-nCAT compositions disclosed herein in biomedical applications, gelatin-based, stable S-nCAT/gelatin hydrogels (termed herein “S-nCAGE”) were synthesized under oxidative environments. These compositions showed desired tissue mimicking mechano-physical properties, properties often unseen in conventional gelatin-based hydrogels. The covalent conjugation of nitrocatecholic domains with gelatin backbone, forming the hydrogel networked, appears to explain the uncompromised chemical behavior of catechol moieties (despite having nitro substitutions). These material properties provide hydrogels with excellent wet tissue adhesive properties, adhesive properties that are significantly higher than those observed with conventional clinical sealants. Interestingly, due to the nitro induced aromatic suturing of nitrocatecholic domains, S-nCAT assured the delocalization of pi-electron along the macromolecular backbone and introduced electroconductive nature to the S-nCAGE, material properties which then facilitate conductive tissue regeneration.

To demonstrate the ability of such hydrogel embodiments of the invention to provide/generate an electroconductive microenvironment, these engineered hydrogels were exposed to mouse muscle myoblasts. These studies showed that the compositions provide a microenvironment having increased electroconductivity as well as in vitro cytocompatibility, with the cells exhibiting increased cellular proliferation and metabolic activity as compared to cells grown in the absence of such compositions. In view of the possible usage of S-nCAGE in biomedical applications. the cytocompatibility and biodegradability of the engineered hydrogel was observed in vivo with subcutaneous implantation in rats. The methods and materials disclosed herein open new opportunities for designing materials via the introduction of new constellations of chemical functionalities on biological small molecules.

The invention disclosed herein has a number of embodiments. Embodiments of the invention include compositions of matter comprising Dopamine and/or Dopamine-like molecules having a nitro (—NO₂) moiety coupled to the aromatic ring (e.g., “nDopa”). Such embodiments include, for example, a composition of matter comprising a hydrogel and a polymer comprising dopamine having a nitro moiety coupled to an aromatic ring. In certain embodiments of the invention, the nDopa (or dopamine like molecule) further comprises a methacrylate moiety coupled to the aromatic ring (e.g., “nMAD”). Typically such methacrylate moieties in these compositions are used to form a polymeric backbone on which nDopa or the like is disposed so as to facilitate chemical suturing of aromatic domains of nDopa and like molecules (see, e.g. FIG. 1). Illustrative working embodiments in the invention include compositions where the polymeric backbones form sutured-nitrocatecholic strands of nDopa polymers (“S-nCAT”). Such compositions typically comprise a gelatin or the like that forms a S-nCAT/hydrogel (“S-nCAGE”). In certain embodiments of the invention, the hydrogel and the nDopa polymers are coupled (e.g., chemically cross linked) together.

The compositions of the invention can include other agents. For example. certain compositions of the invention include a pharmaceutical excipient selected from the group consisting of a preservative, a tonicity adjusting agent, a detergent, a viscosity adjusting agent, a sugar and a pH adjusting agent. Certain compositions of the invention include one or more therapeutic agents such as an anti-inflammatory agent, an agent that modulates coagulation, an antibiotic agent, a chemotherapeutic agent or the like. Certain compositions of the invention include other agents such as live mammalian cells.

In certain compositions of the invention, a constellation of chemical moieties are selectively disposed on the S-nCAGE hydrogel such that it exhibits one or more desirable material properties. For example, in some embodiments for the invention, the hydrogel exhibits an ultimate strength of at least 50 kPa; and/or a tensile toughness of at least 1×104 Jm⁻³): and/or a tensile modulus of at least 40 kPa; and/or a Young's modulus of at least 100 kPa; and/or adhesive energies of at least 5 or 10 Jm⁻²; and/or ionic conductance of at least 1×10⁻³ S/m; and/or an adhesive strength of at least 10, 20, 30 or 40 kPa under physiological conditions. Embodiments of the invention also include methods of making the compositions disclosed herein. For example, embodiments of the methods of making a S-nCAT/gelatin hydrogel comprise forming/combining together: sutured-nitrocatecholic strands of nDopa polymers; and gelatin such that a hydrogel is formed. In typical embodiments of these methods, the methods use selected conditions and materials including molecules having a constellation of chemical moieties that are disposed on the S-nCAGE such that the hydrogel exhibits one or more selected materials properties such as those discussed above in this paragraph.

The compositions of the invention have a number of biomedical uses. For example, embodiments of the invention include methods of adhering a first wet tissue to a second wet tissue (e.g., in vivo at a site of injury or trauma) comprising disposing a hydrogel composition disclosed herein between the first wet tissue and the second wet tissue so as to couple the first wet tissue to the second wet tissue, and adhering the first wet tissue and the second wet tissue by using the hydrogel as an adhesive composition. In certain embodiments of these methods, moieties in the adhesive hydrogel are covalently crosslinked via a cross linking process. In some embodiments of these methods, the adhesive composition that is used in the biomedical application comprises: a pharmaceutical excipient selected from the group consisting of a preservative, a tonicity adjusting agent, a detergent, a viscosity adjusting agent, a sugar and a pH adjusting agent; and/or one or more therapeutic agents such as an anti-inflammatory agent, an agent that modulates coagulation, an antibiotic agent, a chemotherapeutic agent or the like; and/or a diagnostic agent such as a detectable marker; and/or mammalian cells.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1. Engineering S-nCAGE hydrogel with sutured nitrocatecholic building block. (a) Chemical synthesis of nitro-functionalized methacrylated dopaminc (nMAD) from a neurotransmitter molecule, dopamine. (b) 1H-NMR spectra of dopamine derivatives demonstrating sequential functionalization of catecholic moieties. (c,d) Synthesis of sutured-nitrocatacholic strands (S-nCAT) skeleton and its corresponding 1H-NMR spectra. (e) Schematically illustrated S-nCAGE hydrogel framework comprised of S-nCAT and gelatin macromolecule. (f) Covalent and noncovalent molecular interactions in S-nCAGE, forming a 3D hydrogel network. The term “sutured” in this context refers to the uniting of chemical moieties/parts in some pattern akin to a row of stitches holding together the edges of a surgical incision. As shown in FIGS. 1 and 3, this term is used to illustrate the material property of compositions where strands of a polymer comprising nDopa are united using the chemical properties of such moieties/parts.

FIG. 2. Physical properties of S-nCAGE hydrogel. (a) Ultimate strength, (b) stretchability, (c) tensile toughness, and (d) tensile modulus of S-nCAGEx hydrogels synthesized with S-nCATx having different concentrations of nitrocatecholic moieties (x represents the concentration of nitrocatecholic moieties). (e) Representative image of twisting (i-v) and knot formation (vi) with engineered tough S-nCAGE₁ hydrogel. (f) Compressive modulus of S-nCAGEₓ hydrogels. (g) Cyclic compression and (h) corresponding energy loss related to S-nCAGE₁ hydrogel derived with S-nCAT₁. (i) In vitro enzymatic degradation of engineered S-nCAGEₓ hydrogels.

FIG. 3. Multifunctionality of S-nCAGE hydrogel. (a) Schematic illustration along with pictorial representation of the wound closure test setup with S-nCAGE hydrogel over porcine skin. Stretched engineered hydrogel upon applied force while adhered to the wet tissue surface. (b) Interfacial adhesive energy and (c) adhesive strength of the engineered hydrogels over porcine skin in comparison with commercial glues. (d) Interfacial adhesive energy of the S-nCAGE hydrogel when applied on different wet tissue surfaces. (e) Catechol mediated tissue-material interfacial interactions (covalent and noncovalent) of S-nCAGE hydrogel. (f) Aromatic pi-electron delocalization mechanism in S-nCAT. (g) Conductivity of gelatin, p-CAGE₁ (synthesized with p-CAT₁), and S-nCAGE₁ (synthesized with S-nCAT₁) demonstrating the role of nitro-group to introduce conductivity in the hydrogels. (h) Conductivity of S-nCAGEₓ hydrogels prepared with increasing nitrocatecholic concentrations in S-nCATₓ.

FIG. 4. In vitro and in vivo biocompatibility of the engineered biomaterial. Representative (a) live/dead and (b) F-actin/DAPI stained images of C2C12 cells (mouse myoblasts) seeded underside of the transwell permeable supports with hydrogels synthesized with S-nCAT₁ (S-nCAGE₁) and p-CAT₁ (p-CAGE₁) after 1 and 5 days post-seeding (scale bars=100 μm). (c) Quantitative analysis of C2C12 cell viability at days 1, and 5 post-seeding. (d) Quantitative analysis of metabolic activity, relative fluorescence units (RFU), using a PrestoBlue assay at days 1, and 5 post seeding. (e,f) In vivo biodegradation of engineered hydrogels synthesized with S-nCAT/gelatin (S-nCAGE) and monomeric nMAD/gelatin using a rat subcutaneous model and their representative images. (g) Hematoxylin and eosin (H&E), (h) Masson's Trichrome (MT), and (i) immunofluorescence (IF) staining from hydrogel/tissue interfaces at days 7, and 28 post-implantations (scale bars=100 μm). Error bars indicate standard error of the means, asterisks mark significance levels of p<0.0001 (****)).

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the aspects of the techniques and procedures described or referenced herein are well understood and commonly employed by those skilled in the art. The following provides illustrative embodiments of the invention. All publications mentioned herein are incorporated herein by reference to disclose and describe aspects, methods and/or materials in connection with the cited publications.

The introduction or substitution of chemical moieties on bioactive small molecules can alter their electronic behaviors and trigger new molecules with enhanced functional properties. As discussed below, nitro groups are chemical moieties that are very useful to selectively alter biomolecule functionalities. With a large deficit of negative charge, nitro groups are of biological importance and have proven impact in a number of contexts including in chemotherapy. The nitro functionality/moiety/group, with its strong electron-withdrawing capability, can actively modulate electron density of organic moieties and regulate cellular biochemical reactions. However, the exact chemistry of nitro functionalities and their impact in introducing physiologically relevant macroscopic properties in biomolecules such as those that comprise molecular skeletons is unknown.

As disclosed herein, we have manipulated the electronic density of a naturally derived neurotransmitter molecule, dopamine (Dopa), by using nitro-and other moieties/functionalities to create new macromolecular structures. For example, while the introduction of nitro groups facilitated the chemical suturing of aromatic domains of nitrodopamine molecules, the introduction of methacrylated group in nDopa further provided an additional polymeric backbone to engineer the sutured-nitrocatacholic strands, named herein “S-nCAT”. We further show that the methods and materials disclosed herein can introduce biologically relevant macroscopic multifunctionalities while incorporated in biomaterials designed with biomacromolecules like gelatin and are useful in diverse biomedical applications.

As disclosed below, we discovered that the addition of a chemically strong electron withdrawing nitro group easily perturbed the aromatic electron density of nitrocatecholic domain of dopamine and enabled the suturing of nitrocatechol moieties to regain its aromaticity through radical transfer mechanism. To introduce S-nCAT in biomedical applications, gelatin-based, stable S-nCAT/gelatin hydrogel (S-nCAGE) was then created under oxidative environments. Such compositions showed a number of highly desirable tissue mimicking mechano-physical properties, properties often unseen in gelatin-based hydrogels. Meanwhile, covalent conjugation of nitrocatecholic domains with gelatin backbone. forming the hydrogel networked, explained the uncompromised chemical behavior of catechol moieties despite having nitro substitution.

We further demonstrated the excellent wet tissue adhesive properties of S-nCAGE, adhesive properties which are significantly higher than conventional clinical sealants. Interestingly, due to the nitro induced aromatic suturing of nitrocatecholic domains, S-nCAT assured the delocalization of pi-electron along the macromolecular backbone and introduced electroconductive nature to the S-nCAGE, utmost required for the conductive tissue regeneration. S-nCAGE further exhibits ability to provide and electroconductive microenvironment when this engineered hydrogel was exposed to mouse muscle myoblasts, data which shows the in vitro cytocompatibility of such compositions (e.g., which provide for with increased cellular proliferation and metabolic activity). In order to further demonstrate the usefulness of S-nCAGE in biomedical applications, the cytocompatibility and biodegradability of this engineered hydrogel was confirmed in vivo with subcutaneous implantation in rats.

The novel approaches for modifying biomolecules as disclosed herein provides a foundational platform for designing new types of biomacromolecules by introducing chemical functionality in small biomolecules. This chemical functionality, such as addition of nitro moieties to biomolecules is useful to tune the electronic properties of biological small molecules and. in this way, opens new opportunities for developing a wide variety of biomaterials useful in medical science.

The invention disclosed herein has a number of embodiments. Embodiments of the invention include compositions of matter comprising Dopamine having a nitro (NO2) moiety coupled to an aromatic ring (“nDopa”). Such compositions of matter typically comprise a hydrogel and a polymer comprising dopamine having a nitro (NO₂) moiety coupled to an aromatic ring (“nDopa”). In certain embodiments of the invention, the nDopa further comprises one or more functionalized moieties, for example a methacrylate moiety coupled to the aromatic ring (“nMAD”). In certain of these compositions, these methacrylate or other moieties are coupled so as to form a polymeric backbone on which nDopa is disposed and to facilitate chemical suturing of aromatic domains of nDopa molecules on the polymeric backbones.

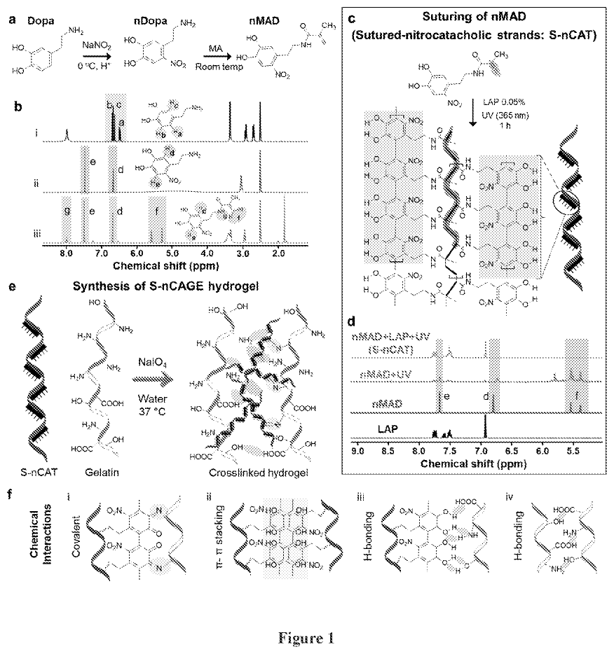

In certain compositions of the invention, the polymeric backbones form sutured-nitrocatecholic strands of nDopa polymers (“S-nCAT” as shown in FIG. 1). In certain embodiments, the composition comprises a hydrogel agent such as gelatin so as to form a gelatin-based. S-nCAT/gelatin hydrogel (“S-nCAGE”) composition. Certain compositions of the invention comprise a pharmaceutical excipient selected from the group consisting of a preservative, a tonicity adjusting agent, a detergent, a viscosity adjusting agent, a sugar and a pH adjusting agent. Certain compositions of the invention comprise one or more therapeutic agents selected from an anti-inflammatory agent, an agent that modulates coagulation, an antibiotic agent, a chemotherapeutic agent. Certain compositions of the invention comprise mammalian cells. In illustrative compositions of the invention, and S-nCAGE hydrogel exhibits one or more of the following materials properties: an ultimate strength of at least 50 kPa; a tensile toughness of at least 1×10⁴ Jm⁻³); a tensile modulus of at least 40 kPa; a Young's modulus of at least 100 kPa; adhesive energies of at least 5 or 10 Jm⁻²; ionic conductance of at least 1×10⁻³ S/m; and/or an adhesive strength of at least 10, 20, 30 or 40 kPa. In certain embodiments of the invention moieties in an ingredient in the composition (e.g., S-nCAGE moieties) are covalently crosslinked.

Embodiments of the invention include methods of making the compositions disclosed herein. For example, embodiments of the invention include methods of making a hydrogel composition comprising sutured-nitrocatecholic strands of nDopa polymers, the method comprising combining together a hydrogel and polymers comprising a polymeric backbone comprising nDopa disposed on the polymeric backbone so as to facilitate chemical suturing of aromatic domains of nDopa molecules: wherein the hydrogel and the polymers are combined so as to form sutured-nitrocatecholic strands of nDopa polymers within the hydrogel composition. Polymers useful to form hydrogels in compositions of the invention include hyaluronic acid, chitosan, heparin, alginate, gelatin and fibrin, as well as polyvinyl alcohol, polyethylene glycol, sodium polyacrylate, acrylate polymers and copolymers thereof. In certain embodiments of the invention, the compositions under an oxidative environment (e.g. an environment comprising periodate ions). In certain embodiments of the invention, the nDopa used in the method is selected to comprise one or more functionalized moieties, for example a methacrylate moiety coupled to the aromatic ring (“nMAD”). Certain methods of making the compositions disclosed herein further comprise including in the hydrogel composition: a pharmaceutical excipient selected from the group consisting of a preservative, a tonicity adjusting agent, a detergent, a viscosity adjusting agent, a sugar and a pH adjusting agent; one or more therapeutic agents selected from an anti-inflammatory agent, an agent that modulates coagulation, an antibiotic agent, a chemotherapeutic agent: and/or mammalian cells.

Embodiments of the invention also include methods of adhering a first wet tissue to a second wet tissue comprising disposing an S-nCAGE composition disclosed herein between the first wet tissue and the second wet tissue so as to couple the first wet tissue to the second wet tissue, and adhering the first wet tissue and the second wet tissue by using the S-nCAGE as an adhesive composition. Typically in these methods, the first wet tissue and the second wet tissue are adhered in vivo. In certain embodiments of the invention, the adhesive composition comprises: a pharmaceutical excipient selected from the group consisting of a preservative, a tonicity adjusting agent, a detergent, a viscosity adjusting agent, a sugar and a pH adjusting agent; and/or one or more therapeutic agents such as an anti-inflammatory agent, an agent that modulates coagulation, an antibiotic agent, a chemotherapeutic agent or the like: and/or mammalian cells. Further aspects and embodiments of the invention are discussed in the following sections.

Dopamine (Dopa), a naturally derived neurotransmitter molecule^{[4]}, also resembles with the key moiety in mussel foot proteins (catechol)^{[11]} and has been intensely utilized to develop adhesive biomaterials with diverse clinical importance^{[12]}. In view of the biological relevance of multifunctional catecholic moieties, herein, Dopa skeleton was chemically modified with a nitro-group to modulate the electronic properties of the aromatic ring^{[13]} (FIG. 1a). To use the nitro incorporated dopamine (nDopa) moiety for designing functional biomaterials, it was further covalently conjugated with a methacrylate group through an amide linkage (FIG. 1a). Sequential synthesis of the methacrylate nitro-dopamine (nMAD) from Dopa was characterized with proton nuclear magnetic resonance (¹H-NMR) spectroscopy which showed the substitution of hydrogen atoms (Hₐ) in the aromatic catechol moieties (FIG. 1b-i,ii) along with the introduction of characteristic methacrylic (H_{f}) and amide hydrogen (H_{g}) peaks in the ¹H-NMR spectrum (FIG. 1b-iii). For instance, while parent Dopa molecule contained spin coupled three C—H peaks (Hₐ-doublet of doublet, H_{b}-doublet, H_{c}-doublet), introduction of nitro-group restricted the aromatic proton couplings and presented two singlet C—H proton peaks (H_{d} and Hₑ) in the aromatic region (FIG. 1b-i,ii). Furthermore, introduction of strong electron withdrawing nitro-group changed the catecholic-electronic environment in the nDopa molecule which caused the shifting of aromatic C—H proton peaks (H_{d} and Hₑ) in the ¹H-NMR spectrum (FIG. 1b-ii). Meanwhile, methacrylic proton peaks (═C—H_{f}) at 5.3 and 5.6 ppm, respectively, confirmed the formation of nMAD through an amide linkage (FIG. 1b-iii). The presence of amide linkage was further revealed with ¹H-NMR spectroscopy, exhibiting a signature triplet —N—H (H_{g}) peak at 8.0 ppm (FIG. 1b-iii) which disappeared through the solvent exchange mechanism with D₂O. Meanwhile, the change in color for native aqueous Dopa solution after 1 h at ambient conditions, confirmed the self-oxidation of catechol moieties, facilitating the formation of toxic polymeric skeletons^{[10]}. This was also observed for methacrylated dopamine (MAD) in 24 h. However, the presence of electron withdrawing nitro-functionality. which reduced the catecholic electron density in nMAD, prevented its self-oxidation at ambient conditions even after 7 days.

Chemical contribution of nitro-functionality towards the formation of a new macromolecular structure from monomeric nMAD is presented in FIG. 1c. Herein, electron-deficient aromatic building blocks (nMAD) were treated with UV light (365 nm) in the presence of a photoinitiator molecule, lithium phenyl-2,4,6-trimethylbenzoylphosphinate (LAP), to synthesize a sutured-nitrocatacholic strands (S-nCAT) through an electron transfer radical mechanism (FIG. 1c). While reactive methacrylate groups of nMAD formed polymeric —C—C— covalent bonds (FIG. 1c, red shaded), electron deficient nitrocatecholie moieties interacted with each other and formed a novel macromolecular structure through the suturing of aromatic rings (FIG. 1c, gray shaded). Formation of polymeric —C—C— covalent bonds were confirmed with ¹H-NMR, showing the consumption of methacrylic hydrogens in S-nCAT spectrum (FIG. 1d, H_{f}-red shaded). Suturing of nitrocatecholic moieties during the LAP-induced UV-photocrosslinking process was also revealed with ¹H-NMR, showing the consumption of aromatic hydrogens of nMAD (FIG. 1d, H_{d} and Hₑ-gray shaded), forming a novel macromolecular structure, S-nCAT. Mechanistically, such aromatic ring suturing could recover and extend the aromatic stability of the electron-deficient nitrocatechol moieties upon delocalization of pi-electrons along the sutured molecular skeleton^{[14]}. To demonstrate the involvement of the nitro functionality in aromatic ring suturing, MAD molecules were treated with identical chemical reaction conditions to form polymeric catechol structure (p-CAT) and analyzed with ¹H-NMR. Consumption of methacrylic hydrogens with unaltered aromatic hydrogen peaks of MAD building blocks confirmed the formation polymeric —C—C— covalent bond, while catechol rings remained unreacted.

In view of the biological importance of both nitro and catecholic moieties, as synthesized novel macromolecular structure (S-nCAT) was used to design a new hydrogel for biomedical applications. For this, as synthesized S-nCAT was covalently linked with gelatin molecules through a schiff base reaction^{[12c]}, forming the primary molecular skeleton of the biomaterial (FIG. 1e). Chemical interactions in the engineered S-nCAT/gelatin hydrogel (S-nCAGE) networks are shown in FIG. 1f, which include both covalent and noncovalent bondings between the macromolecules. Specifically, amine groups of gelatin backbone (lysine amino acid residue) chemically interacted with nitrocatechilic moieties of S-nCAT in an oxidative environment, forming a stable hydrogel structure through covalent linkage (FIG. 1f-i). Consumption of these amine functionalities of gelatin backbone was analyzed by ¹H-NMR, showing the absence of —C—H proton (δ 2.75, related to —CH₂ of lysine residue) for completely crosslinked hydrogel. To reveal the amine mediated covalent crosslinking mechanism. oxidative environment of the hydrogel was regulated with periodate concentrations which could control the reactivity of nitrocatecholic moieties towards the amine groups and further analyzed with ¹H-NMR. For instance, while partially oxidized catechol moieties were covalently linked with 25% amine of gelatin backbone, further increase in oxidative environment resulted in complete consumption of amine groups of gelatin macromolecules. Such facile interactions between amine and oxidized nitrocatechol moieties demonstrated the unaltered chemical behavior of catecholic domains present in S-nCAT. Meanwhile, apart from the covalent interactions (FIG. 1f-i), engineered hydrogel (S-nCAGE) also comprised of noncovalent interactions in its molecular network (FIG. 1f-ii-iv). These include pi-pi stacking of sutured-nitrocatecholic moieties (FIG. 1f-ii), hydrogen bonding interactions between nitrocatecholic moieties and hydrophilic groups of gelatin (FIG. 1f-iii), and hydrogen bonding between the gelatin macromolecules (FIG. 1f-iv).

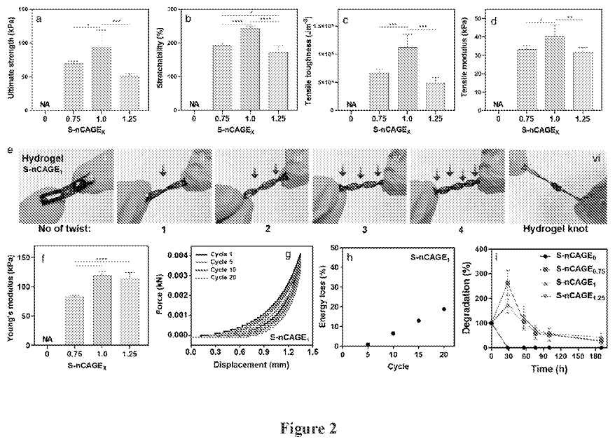

Stability of a biomaterial in physiological conditions is crucial for its usage in surgical and clinical treatment procedures^{[12c, 15]}. However, naturally derived gelatin macromolecules, decorated with hydrophilic functional groups, are incapable to form a biocompatible hydrogel with adequate mechanical properties without further chemical modification^{[12a, 16]}. Meanwhile, the engineered S-nCAGE hydrogel, with multiple covalent and noncovalent interactions in its molecular framework, showed excellent mechanical properties (FIG. 2a-d) and stability in an aqueous solution at 37° C. over time. To study the effect of chemico-physical crosslinking on the mechanical properties of S-nCAGE, hydrogels were synthesized with S-nCATₓ having different concentrations of nitrocatecholic moieties (x represents the concentration of nitrocatecholic moieties). The engineered hydrogel prepared without S-nCAT is name as S-nCAGE₀, whereas S-nCAGEₓ hydrogels synthesized with S-nCATₓ (x=0.75, 1, 1.25% w/v nMAD) were described as S-nCAGE_{0.75}, S-nCAGE₁, and S-nCAGE_{1.25}, respectively. Rapid dissolution of the physically gelled gelatin hydrogel without S-nCAT (S-nCAGE₀, control sample) in water at 37° C. described the lack of molecular integrity, demonstrating the difficulties of its usage in medical procedures. In contrast, incorporation of S-nCAT showed significant improvement in the mechanics of engineered hydrogels when tested with tensile perturbation (FIG. 2a-d). For instance, the ultimate strength for the S-nCAGE hydrogels synthesized with S-nCAT_{0.75} and S-nCAT₁ were improved from ˜72 to ˜100 kPa. respectively (FIG. 2a). Such improvement in the mechanical strength with increasing nitrocatecholic environment in S-nCAT could be related to the enhanced covalent and noncovalent interactions, as described in FIG. 1f. Meanwhile, the S-nCAGE_{1.25}, which was synthesized with S-nCAT_{1.25}, prevented the injectability of the hydrogel and caused material loss during the hydrogel formulation, thus, decreased the ultimate strength. Incorporation of S-nCATₓ skeleton also demonstrated improvement in the extensibility of S-nCAGEₓ hydrogel (FIG. 2b). which was significantly higher compared to the previously developed elastic biomaterials with poly (ethylene glycol), and other macromolecules^{[12a, 15, 17]}. Stretchability of S-nCAGE₁ up to ˜250% (FIG. 2b) could be explained with enhanced noncovalent interactions (FIG. 1f-ii-iv) in the hydrogel matrix^{[18]}. This is comparable with the elasticity of pulmonary artery and other native elastic tissues^{[17b, 19]}. Along with the stretchability, active noncovalent interactions introduced toughness^{[18]} to the S-nCAGE hydrogel with a maximum toughness of ˜1.3×10⁵ Jm⁻³ (FIG. 2c), essential for the treatment of dynamic organs to support the physiological tissue deformation. Furthermore, the engineered hydrogel also owned biologically relevant tensile modulus in the range of 30-45 kPa (FIG. 2d), which can maintain the intrinsic mechanical properties of the native tissues.

Physical appearance along with the flexibility of the engineered hydrogel upon mechanical perturbation (twisting, knot formation and stretching) is pictorially presented in FIG. 2e. Although, in most of the cases, gelatin backbone formed brittle and nonstretchable biomaterials^{[12a, 15, 20]}, robust and dynamic molecular framework of S-nCAGE comprised of dense covalent and noncovalent interactions enabled the twisting (FIG. 2e-i-v) and knot formation (FIG. 2e-vi), followed by manual stretching. Elastic deformation of S-nCAGE hydrogels were further characterized with compression test which showed a maximum Young's modulus over ˜120 kPa (FIG. 2f), mimicking the mechanics of various biological tissues. To demonstrate noncovalent interaction mediated dynamic molecular rearrangements, S-nCAGE; hydrogel was allowed to undergo cyclic compression test (FIG. 2g) which exhibited an energy loss of ˜20% upon completion of 20 cycles (FIG. 2h).

The presence of hydrophilic hydroxyl groups in catechol moieties resulted in diffusion of water molecules^{[21]} and caused swelling of the crosslinked hydrogel. For instance, while S-nCAGE_{0.75} swelled up to ˜160%, S-nCAGE_{1.25} hydrogel, with increased nitrocatecholic environment, resulted in a maximum swelling in the order of ˜280%. Meanwhile, the enzymatic degradation profiles for all hydrogel formulations were similar due to the presence of same amount of gelatin backbone which actively interacted with collagenase (FIG. 2i). To highlight the contribution of both —C—C— backbone and sutured-nitrocatecholic moieties of S-nCAT on improving the stability of S-nCAGE, hydrogels were prepared with monomeric nMAD and gelatin which showed faster degradation as compared to the S-nCAGE hydrogels using similar enzymatic environment.

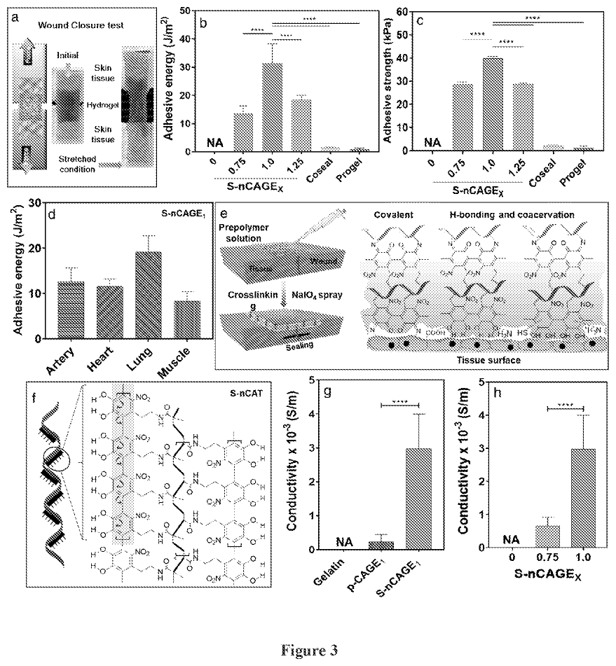

Biologically, in addition to the major role in the central nervous system as a neurotransmitter^{[4]}, catechol moieties in mussel foot proteins^{[11]} also control the interfacial interactions of marine mussels with the wet surfaces^{[12c]} to facilitate their adherence underwater. Therefore, to evaluate the interfacial activities of S-nCAT macromolecules with nitrocatecholic moieties, the adhesion of S-nCAGE hydrogels to wet porcine skin tissue surface was studied using a standard wound closure (ASTM F2458-05) test (FIG. 3a). While mechanically unstable gelatin hydrogel without S-nCAT (S-nCAGE₀) is incapable to hold the tissues, engineered S-nCAGEₓ hydrogels prepared with S-nCATₓ showed strong tissue adhesive property (FIG. 3b-d), reflecting unaltered chemical behavior of catecholic moieties after nitro-functionalization. With increasing the nitrocatecholic concentrations in S-nCAT (S-nCAT_{0.75} to S-nCAT₁), both interfacial adhesion energy and adhesion strength of the engineered hydrogels significantly enhanced, reaching values much higher than the commercial glues (FIG. 3b,c). For instance, the adhesive hydrogel prepared with S-nCAT₁ demonstrated ˜20, and ˜35-fold increase in the interfacial adhesion energy over the wet porcine skin tissue, respectively, as compared to the commercial glues, coseal, and progel (FIG. 3b). Similarly, the adhesive strength of S-nCAGE₁ was ˜40 kPa. which was ˜19, and ˜15.7 times higher than coseal, and progel (FIG. 3c), respectively, demonstrating the potential use of the engineered hydrogel as a bioadhesive for sealing of soft tissues. Meanwhile, decreased in adhesive behavior of S-nCAGE_{1.25} was due to the material loss upon physical gelation during the application of the hydrogel over the tissue surface. To investigate the interfacial adhesive characteristics of the engineered hydrogel to different tissue surfaces, wound closure tests were performed using various wet tissues including artery, heart, lung, and muscle (FIG. 3d). Interestingly. for all tissue types, the engineered hydrogel showed efficient adherence with adhesive energies in the range of ˜10-20 Jm⁻², which was much higher than the commercial glues (˜1-1.6 Jm⁻²), shown in FIG. 3b. Functional covalent and noncovalent tissue-material interfacial interactions through the catecholic moieties of S-nCAT are schematically presented in FIG. 3e, which include covalent bonding through schiff base reaction, hydrogen bonding, and coacervation mechanisms^{[12c]}.

Biologically. even though the catechol derivatives possess significant role as cellular chemical messengers, catechol-derived biomaterials lack conductivity which is important for the regeneration of electro-conductive tissues^{[22]}. Therefore, in most of the cases, along with the catechol moieties, a conducting constituent is incorporated in the scaffold to provide a suitable chemical microenvironment for tissue regeneration^{[23]}. Interestingly, S-nCAT with nitro-mediated sutured aromatic moieties enabled the extended pi-electrons delocalization (FIG. 3f) and introduced biologically relevant conductive behavior to the S-nCAGE hydrogel (FIG. 3g,h). Thus, S-nCAT, our new macromolecular skeleton, could serve both conductive and dopamic microenvironment to support both adhesion and growth of electro-conductive tissues. The pi-electron delocalization mechanism though the sutured aromatic ring of S-nCAT skeleton, enabling the conductive property in the engineered hydrogel, is schematically presented in FIG. 3f. To demonstrate the role of sutured-nitrocatecholic rings to introduce conductivity in the S-nCAGE hydrogel network. conductivity measurements was performed with p-CAGE₁ (synthesized with p-CAT₁, without nitro-group), and S-CAGE₁ (synthesized with S-nCAT₁, with nitro-group) hydrogels. While gelatin hydrogel showed no conductivity, incorporation of p-CAT₁ (unsutured catechol moieties) demonstrated ionic conductance in the order of ˜2.3×10⁻⁴ S/m (FIG. 3g). In contrast, introduction of S-nCAT₁ (with sutured catechol moieties, FIG. 1c) increased the conductivity of the engineered hydrogel by ˜13 times (˜2.98×10⁻³ S/m) (FIG. 3g). Such enhanced electro-conductive behavior of S-nCAGE hydrogel could be due to both electron delocalization mechanism and ionic conductance. Meanwhile, the engineered hydrogels prepared with increasing concentration of nitrocatecholic building blocks in S-nCAT enriched the hydrogel network with sutured aromatic strands, which subsequently improved the conductivity of the hydrogels (FIG. 3h). Introduction of such electroconductive microenvironments by electro-manipulation of chemical substituents in the small bioactive molecules could be useful for designing materials for different biomedical applications including engineering adhesive and organic bioelectronics^{[24]}.

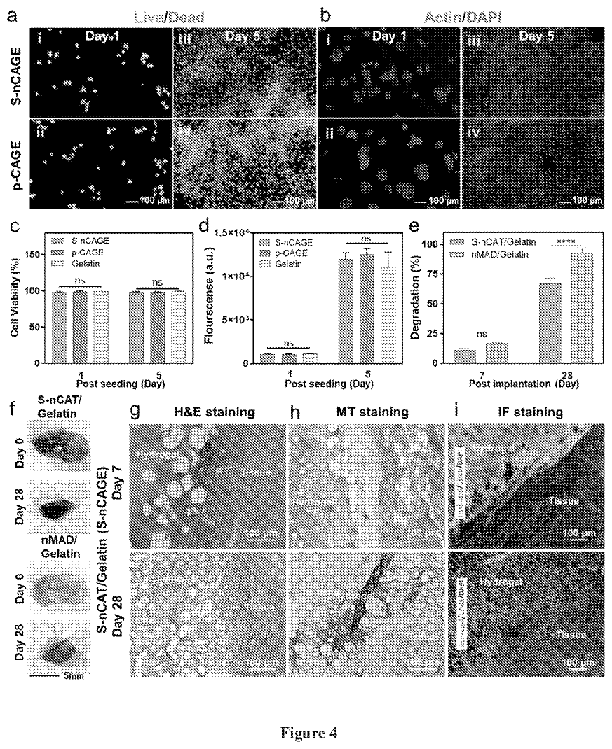

To demonstrate the biocompatibility of the engineered multifunctional S-nCAGE hydrogel for clinical usages, cytotoxicity was evaluated with C2C12 cells (mouse muscle myoblast) in transwell plate. Although, both catechol and nitrocatechol are known in biological system^{[10, 12c]}, to understand the cellular response to the chemically modified catecholic environments, hydrogels were synthesized with MAD, nMAD, p-CAT, and S-nCAT, respectively, and compared with gelatin hydrogel prepared through a physical gelation process (FIG. 4a-d). For all formulations, engineered hydrogels supported the viability of C2C12 cells which was comparable with gelatin hydrogel, confirming the in vitro cytocompatibility of the synthesized materials (FIG. 4a). Quantitative cell viability above 95% up to 5 days revealed the absence of toxic chemical environment provided by all the hydrogels (FIG. 4c). Furthermore, the morphological changes of C2C12 cells cultured under different catecholic environments was evaluated with F-actin/DAPI staining, which showed spreading of C2C12 over 5 days of incubation comparable to the gelatin hydrogel (FIG. 4b). In addition, significantly enhanced relative fluorescence intensity from day 1 to day 5 in PrestoBlue assay, quantitatively described the increased cellular proliferation and metabolic activity of the C2C12 cells (FIG. 4d). Therefore, all together these results suggest that the developed multifunctional S-nCAGE hydrogel was biocompatible, confirming their use for biomedical applications.

To evaluate the in vivo degradation and biocompatibility of the engineered hydrogel, subcutaneous implantation was performed in rats (FIG. 4e-i). For this. hydrogels were prepared with S-nCAT and monomeric nMAD to understand the effect of polymeric —C—C— covalent backbone and sutured-nitrocatecholic strand in biodegradation and in vivo tissue ingrowth. Due to the absence of these additional bonding interactions in the hydrogel network prepared with monomeric nMAD, nMAD/Gelatin hydrogel showed faster degradation rate after 28 days of implantation as compared to the S-nCAGE hydrogel (FIG. 4e,f). Meanwhile, histological studies on the explanted hydrogels at days 7, and 28 post-surgery showed significant amount of cell infiltration in Hematoxylin and cosin (H&E) and Masson's Trichrome (MT) stained samples (FIG. 4g,h), demonstrating the biocompatibility and biointegration of the materials with the host tissue with no significant fibrosis. In addition, the presence of macrophage (CD68) within the normal physiological range confirmed the non-inflammatory responses by the host upon subcutaneously implantation of hydrogels prepared with S-nCAT (S-nCAGE) and monomeric nMAD (FIG. 4i). Thus, in vivo studies suggest that the engineered hydrogel with nitro-group mediated sutured aromatic backbone, which introduced biologically relevant multifunctional properties, maintained the biocompatibility, and could be used for biomedical applications.

### Experimental Section

Materials. Gelatin from porcine skin type A, methacrylate anhydride, sodium tetraborate, sodium bicarbonate, sodium nitrite, sodium periodate, and, lithium phenyl-2,4,6-trimethylbenzoylphosphinate (LAP) were purchased from Sigma Aldrich (USA) and used without any further purification. Dopamine hydrochloride, hydrochloric acid solution, ethyl acetate, hexane, tetrahydrofuran, Dulbecco's phosphate-buffered saline (DPBS) were purchased from Thermo Fisher Scientific (USA). Dulbecco's modified eagle medium (DMEM) was purchased from ATCC (USA) and supplemented with 10% fetal bovine serum (CORNING, USA) and 1% penicillin/streptomycin (Life Technologies, USA). The commercial live/dead™ kit (calcein AM and ethidium homodimer-1), and AlexaFluor 488 F-Actin/DAPI were purchased from Invitrogen (Carlsbad, CA). PrestoBlue™ assays was purchased from Life Sciences (USA).

Synthesis of nitro-dopamine. Nitro-dopamine was synthesized according to previously reported nitration of aromatic compounds with further modification^{[25]}. Briefly, 500 mg of dopamine hydrochloride (Dopa) and 630 mg of NaNO2 were dissolved in ice cold water followed by addition of 20% sulphuric acid solution under vigorous stirring condition. The reaction was continued for 1 h until the precipitation of yellow nitro substituted dopamine molecules (nDopa). Hereafter, the yellow precipitate was thoroughly washed with cold methanol and water multiple times to remove the unreacted chemicals. Later on, the cleaned nDopa molecules were dried under vacuum for 2 days and stored in room temperature for further use.

Synthesis of methacrylated dopamine (MAD) and methacrylated nitro-dopamine (nMAD). To synthesis MAD, previously reported method was adopted with further modification^{[26]}. Briefly, Dopa (10 g) was dissolved in nitrogen bubbled solution of sodium borate and sodium bicarbonate. Next, 50 ml of methacrylic anhydride (MA) was dissolved in tetrahydrofuran and added to the reaction mixture dropwise under vigorous stirring. The reaction was continued overnight, followed by washing with ethyl acetate for 3 times. Hereafter, the pH of the aqueous phase was reduced to below 2 by adding hydrochloric acid solution and the organic layer of the reaction mixture was collected for further extraction. Next, the volume of the extracted organic layer was reduced by using a rotavapor and mixed with cooled hexane to precipitate MAD. Precipitated MAD was further purified by using cooled hexane, dried under vacuum and stored for further use. To synthesize nMAD, same reaction method was followed with nDopa.

Synthesis of S-nCAT with sutured-nitrocatechol moieties. Macromolecular structures of nitrocatecholic moieties were synthesized using a UV mediated radical reaction. Herein, different concentrations of monomeric nMAD (0.75, 1 and 1.25% w/v) were dissolved in LAP solutions prepared in milliQ water and irradiated with UV light for 1 h. The synthesized S-nCATₓ macromolecules with X=0.75, 1 and 1.25% w/v nMAD were denoted as S-nCAT_{0.75}, S-nCAT₁, and S-nCAT_{1.25}, respectively. During the synthesis, along with the formation of polymeric —C—C— structure from reactive methacrylate group, nitrocatechol moieties of nMAD also interacted to form sutured nitrocatecholic strands. To confirm the involvement of nitro-group in aromatic ring suturing, monomeric MAD was exposed to the same chemical environment to synthesis p-CAT macromolecules.

Synthesis of hydrogel. Hydrogels were synthesized by mixing porcine gelatin (20% w/v) to the as prepared S-nCATₓ macromolecule solutions. Engineered S-nCATₓ/gelatin hydrogel was named as S-nCAGEₓ. S-nCAGEₓ hydrogels synthesized with S-nCAT0.75, S-nCAT₁, and S-nCAT_{1.25}, were named as S-nCAGE_{0.75}, S-nCAGE₁, and S-nCAGE_{1.25}, respectively. Herein, the primary structure of the hydrogel was formed by covalent crosslinking between amine group of gelatin and nitrocatecholic moieties when exposed to oxidative environment with periodate ions. Briefly, to prepare the hydrogel gelatin was dissolved in as prepared S-nCATₓ solution at 37° C. and the homogeneous mixture was allowed for chemical crosslinking with periodate solution (100 μL, 30 mM) at room temperature for 15 min. As gelatin is the only source of amine functionality, formation of covalent linkages was investigated through ¹H NMR by monitoring the consumption of amine groups of gelatin molecules. To form the hydrogel with p-CAT, same synthetic procedure was followed and named as p-CAGE. Meanwhile, for the synthesis of hydrogels with monomeric MAD and nMAD, gelatin molecules were mixed with aqueous solution of MAD and nMAD, followed by same chemical crosslinking procedure described above.

¹H Nuclear magnetic resonance (¹H NMR). For the chemical characterization of synthesized nDopa, MAD, nMAD, S-nCAT, and p-CAT, proton nuclear magnetic resonance (¹H NMR) spectroscopy was performed using a 400 MHz Bruker AV400 spectrometer. All the spectra were recorded at room temperature in DMSO-d₆ and D₂O, depending on the requirement. To perform the deuterium exchange, ¹H NMR samples were prepared in DMSO-d₆ and mixed with 10% v/v D₂O. Particularly for the ¹H NMR of S-nCAT and p-CAT, aqueous solutions of as synthesized macromolecules were freeze dried and dissolve in respective deuterated solvents. To assess the covalent crosslinking density in the hydrogel network, freeze dried hydrogels (before and after crosslinking) were dissolved in DMSO-d₆ and measured for ¹H NMR. In this case, sample were prepared with 10 times dilution and the change in —CH₂ peak (attached with the amine of gelatin from lysine residue) in the ¹H NMR spectra was integrated while peak at δ-7.25 was taken as reference. For all spectra, phase, and base line were corrected and the solvent peak was fixed before the analysis.

Mechanical tests (compression and tensile). For compression test, 100 μl of the hydrogel precursor solution was pipetted into a polydimethylsiloxane (PDMS) cylindrical mold (diameter: 6 mm; height: 2.5 mm) and crosslinked according to the above-mentioned crosslinking procedure. Before each test, precise dimensions of the sample were measured using a digital caliper, carefully placed on the compression plate of an Instron 5943 mechanical tester, and the test was conducted at a rate of 1 mm/min. Compressive modulus of the samples were calculated by measuring the slop of the respective stress-strain curve recorded utilizing Bluchill® 3 software. For tensile test, 100 μl of the hydrogel precursor solution was pipetted into a polydimethylsiloxane (PDMS) rectangular mold (10 mm length, 5 mm width, and 1.5 mm thickness) and crosslinked according to the previously described method. Hereafter, the samples were glued with the tensile adhesive tape and loaded on the Instron's tensile grips. Force-displacement data were recorded while performing quasi-static tensile tests at 4 mm/min. The slop of the strain-stress curve was measured to calculate the tensile modulus of the respective samples. Stretchability and tensile strength of the material were defined by strain and stress at failure point, respectively. Finally, the toughness of the materials was measured from the area under the stress-strain curves.

In vitro wound closure test. To characterize adhesive property of the engineered hydrogels prepared with S-nCATₓ, a standard wound closure test (ASTM F2458-05) was performed using porcine tissues in wet condition. Briefly, in all the cases, rectangular shaped tissues were glued onto the two precut glass slides using Krazy Glue. Then, these tissues were cut from the middle using a single edge cutter blade. Hereafter, 100 μl of the precursor solution was applied over these tissues covering the area of 10 mm×10 mm and crosslinked with above-mentioned crosslinking procedure. Herein, to mimic the physiological body environment, surrounding temperature was maintained at 37° C. Next, the sample was loaded on the Instron 5943 mechanical tester, and the tensile force was applied to the two ends of the tissues with a rate of 4 mm/min. The adhesive strength was measured at the failure point. By measuring the area under the force-displacement curve until the breaking point, interfacial adhesive energy was calculated.

Evaluation of conductivity of the hydrogel. To measure the conductivity of engineered hydrogels synthesized with S-nCAT and p-CAT, samples were prepared in a PDMS mold (10 mm×5 mm×1 mm) and crosslinked according to the previously described method. Herein, a two-probe measuring setup using a potentiostat model 263 A, AMETEK® was used. During the test, as prepared samples were washed with MilliQ water and placed between two copper sheets coated with silver paste and the voltage was applied in 0.05 V increments from −1 to 1 V. The current as a function of voltage was recorded and the conductivity of the materials were calculated by using Ohm's law.

Evaluation of in vitro Swelling Ratio. The swelling behavior of the hydrogels were calculated by measuring the weight of the diffused water inside the matrix while submerged in DPBS. For this, hydrogels were prepared in rectangular shape, weighed (initial weight) and submerged into DPBS at 37° C. Hereafter, the change in weight of hydrogels with time were measured and the swelling was calculated with respect to the initial weights.

Evaluation of in vitro Enzymatic Degradation. In vitro degradation of the engineered hydrogels was measured using collagenase type II solution. For this, crosslinked hydrogels were weighed and exposed to the collagenase type II solution in DPBS (2 μg/ml). In this case, hydrogels were placed in separate wells of a 24 well plate filled and kept at 37° C. The collagenase type II solution was changed every 2 days. The samples were weighed at different time intervals and the degradation was determined using the Equation below.

Degradation (%)=(W−Wₜ)/W₀×100

W₀ is the initial weight, and Wₜ is the weight at time t.

In vitro biocompatibility test. To evaluate the cytocompatibility of the engineered hydrogels, C2C12 (mouse myoblasts) were seeded at 1×10⁴ cells/cm² on the bottom of 24-wells transwell permeable (Costar®, 8 μm PET membrane). Herein, hydrogels were prepared with MAD, nMAD, S-nCAT, and p-CAT. Hereafter, as prepared hydrogel samples were placed into the transwell inserts, and 1 ml of growth medium, DMEM supplemented with 10% fetal bovine serum, and 1% penecilin/streptomyosin was added to each well of the transwell permeable supports. The wellplates were incubated at 37° C. in a 5% CO₂ humidified atmosphere for 5 days and the culture medium was replaced every 48 h.

The viability of C2C12 grown on the bottom of wellplates was evaluated using a live/dead™ viability kit (Invitrogen) according to instructions from the manufacturer. Briefly, cells were stained with 0.5 μL/mL of calcein AM and 2 μL/mL of ethidium homodimer-1 (EthD-1) in DPBS for 20 min at 37° C. Fluorescent imaging was performed at days 1, and 5 post-seeding using an AxioObserver Z7 inverted microscope. Viable and dead cells were determined by green and red color, respectively and quantified using the Image) software. Cell viability was determined as the number of live cells divided by the total number of cells.

The metabolic activity of the cells was assessed at days 1, and 5 post-seeding, using a PrestoBlue™ assay (Life Technologies). Briefly, the cultures of C2C12 were incubated in 400 μL of growth medium containing 10% v/v PrestoBlue reagent for 45 min at 37° C. The resulting fluorescence was measured using a Synergy HT fluorescence plate reader (BioTek).

Meanwhile, to observe C2C12 cell spreading on the bottom of 24-wells transwell permeable supports, fluorescent staining was performed using F-actin and DAPI. Briefly, cell cultures at days 1 and 5 post-seeding were fixed in 4% v/v paraformaldehyde (Sigma) for 15 min, permeabilized in 0.3% w/v Triton X-100 (Sigma) for 5 min, and then blocked with 1% w/v bovine serum albumin (BSA, Sigma) for 30 min. Samples were then incubated with Alexa fluor™ 488 phalloidin for 45 min. After 3 washes with DPBS, samples were further stained with 1 μL/mL DAPI (4′,6-diamidino-2-phenylindole, Sigma) in DPBS for 2 min. Finally, the fluorescent imaging was performed using an inverted fluorescence microscope (Zeiss Axio Observer Z7). Herein, to improve the contrast and visualization the cells, color of the F-actin staining was set to red on the fluorescence microscope.

In vivo subcutaneous implantation in rats. All in vivo studies were approved by the ICAUC (ARC-2021-113) at University of California Los Angeles (UCLA). Male Wistar rats (˜250 g) were purchased from Charles River Laboratories (Boston, MA, USA). Anesthesia was performed by inhalation of isoflurane (˜2%). After anesthesia, eight 1-cm incisions were created on the rats' dorsal skin, and small subcutaneous pockets were made using a blunt scissor. Engineered hydrogels were prepared by using a cylindrical PDMS mold as described before. The hydrogels were lyophilized and sterilized under UV light for 10 min and were implanted into the subcutaneous pockets. The incisions were closed with 4-0) polypropylene sutures (Ad Surgical). At days 7, and 28 post-surgery, the rats were euthanized and half of the hydrogels were harvested with their surrounding tissues while the other half were explanted and weighed to determine the degradation rate.

Histological analyses were carried out on the explanted hydrogels to evaluate the inflammatory responses caused by the implanted hydrogels. After explanation of the hydrogels, they were fixed in 4% paraformaldehyde for 4 hours, and incubated at 4° C. in 15 and 30% w/v sucrose solution, respectively. Samples were then embedded in Optimal Cutting Temperature (O.C.T) compound, frozen in liquid nitrogen, and sectioned using Leica CM1950 cryostat machine. 8-10 μm sections were mounted on positively charged slides using DPX mountant (Sigma) for Hematoxylin and Eosin (H&E) staining, Masson's Trichrome (MT) staining, and ProLong™ Gold antifade reagent (Thermo fisher scientific) for immunofluorescence (IF) staining. The slides were then processed for H&E and MT staining (Sigma) according to manufacturer instructions. IF staining was also performed on mounted samples as previously reported^{[27]}. Anti-CD68 (ab125212) (Abeam) was utilized as a primary antibody, and Goat-anti Rabbit IgG (H+L) conjugated to Alexa Fluor™ 594 (Invitrogen) was used as a detection reagent (secondary antibody). All samples were then stained using 4′,6-diamidino-2-phenylindole (DAPI). Lastly, the fluorescent imaging was performed using ZEISS Axio Observer Z7 inverted microscope.

Data analysis. Data analysis was carried out using a 1- or 2-way ANOVA test with GraphPad Prism 9 software. Error bars represent mean #standard deviation (SD) of measurements (*p<0.05, ** p<0.01, *** p<0.001, and **** p<0.0001).

### CONCLUSION

In conclusion, we demonstrated the synthesis of a novel macromolecular structure by tunning the electronic property of the biologically relevant catecholic building blocks with electron withdrawing nitro functionality. Chemically, incorporation of nitro functionality facilitated the aromatic suturing of nitrocatecholic moieties without compromising the parent chemical reactivity and functional interactions of catecholic moieties. This was further demonstrated with the wet tissue adherence which was significantly higher compared to the surgical sealants. Interestingly, sutured-nitrocatechol moieties introduced conductive characteristic to the engineered hydrogel which could provide both catecholic and conductive environment to support the growth of electro-conductive tissues. In vitro and in vivo studied also confirmed the cytocompatibility of the engineered material, demonstrating its potential use for biomedical applications. We believe, the new approach of designing biomacromolecules by manipulating the electronic density of bioactive small molecules with appropriate chemical substitution and developing multifunctional biomaterial will help to overcome the barrier of engineering materials for medical applications.

### REFERENCES

[1] a) T. L. Nero, C. J. Morton, J. K. Holien, J. Wielens, M. W. Parker, Nature Reviews Cancer 2014, 14, 248; b) J. A. Wells, C. L. Mcclendon, Nature 2007, 450, 1001; c) L. L. Blazer, R. R. Neubig, Neuropsychopharmacology 2009, 34, 126.
[2] C. Bissantz, B. Kuhn, M. Stahl, J Med Chem 2010, 53, 5061.
[3] M. Rahmati, E. A. Silva, J. E. Reseland, C. A. Heyward, H. J. Haugen, Chemical Society Reviews 2020, 49, 5178.
[4] T. J. Crow, G. W. Arbuthnott, Nature New Biology 1972, 238, 245.
[5] T. Nagatsu, Proc Jpn Acad Ser B Phys Biol Sci 2007, 82, 388.
[6] C. Borsari, D. J. Trader, A. Tait, M. P. Costi, J Med Chem 2020, 63, 1908.
[7] H. Szatylowicz, A. Jezuita, T. M. Krygowski, Structural Chemistry 2019, 30, 1529.
[8] D. Olender, J. Żwawiak, L. Zaprutko, Pharmaceuticals (Basel) 2018, 11.
[9] K. Nepali, H.-Y. Lee, J.-P. Liou, J Med Chem 2019, 62, 2851.
[10] Z. Shafiq, J. Cui, L. Pastor-Pérez, V. San Miguel, R. A. Gropeanu, C. Serrano, A. del Campo, Angewandte Chemie International Edition 2012, 51, 4332.
[11] Q. Lin, D. Gourdon, C. Sun, N. Holten-Andersen, T. H. Anderson, J. H. Waite, J. N. Israclachvili, Proceedings of the National Academy of Sciences 2007, 104, 3782.
[12] a) H. Montazerian. A. Baidya, R. Haghniaz, E. Davoodi, S. Ahadian, N. Annabi, A. Khademhosseini, P. S. Weiss, ACS Applied Materials & Interfaces 2021, 13, 40290; b) M. Kharaziha, A. Baidya, N. Annabi, Advanced Materials 2021, 33, 2100176; c) W. Zhang, R. Wang, Z. Sun, X. Zhu, Q. Zhao, T. Zhang, A. Cholewinski, F. Yang. B. Zhao, R. Pinnaratip, P. K. Forooshani, B. P. Lee, Chemical Society Reviews 2020, 49, 433.
[13] O. A. Stasyuk, H. Szatyłowicz, T. M. Krygowski, C. Fonseca Guerra, Physical Chemistry Chemical Physics 2016, 18, 11624.
[14] a) D. J. Babinski, X. Bao, M. El Arba, B. Chen, D. A. Hrovat, W. T. Borden, D. E. Frantz, Journal of the American Chemical Society 2012, 134, 16139; b) Z.-M. Chen, X.-M. Zhang, Y.-Q. Tu, Chemical Society Reviews 2015, 44, 5220.
[15] M. Tavafoghi, A. Sheikhi, R. Tutar, J. Jahangiry, A. Baidya, R. Haghniaz, A. Khademhosseini, Advanced Healthcare Materials 2020, 9, 1901722.
[16] a) G. Yang, Z. Xiao, H. Long, K. Ma, J. Zhang, X. Ren, J. Zhang, Scientific Reports 2018, 8, 1616; b) X. Zhou, Z. Luo, A. Baidya, H.-j. Kim, C. Wang, X. Jiang, M. Qu. J. Zhu, L. Ren, F. Vajhadin, P. Tebon, N. Zhang, Y. Xue, Y. Feng, C. Xue, Y. Chen, K. Lee, J. Lee, S. Zhang, C. Xu, N. Ashammakhi, S. Abadian, M. R. Dokmeci, Z. Gu, W. Sun, A. Khademhosseini, Advanced Healthcare Materials 2020, 9, 2000527.
[17] a) C. Xu, W. Lee, G. Dai, Y. Hong, ACS Applied Materials & Interfaces 2018, 10, 9969; b) J. Qu, X. Zhao, Y. Liang, T. Zhang, P. X. Ma, B. Guo, Biomaterials 2018, 183, 185.
[18] Y. Wang, X. Huang, X. Zhang, Nature Communications 2021, 12, 1291.
[19] a) Y.-N. Zhang. R. K. Avery, Q. Vallmajo-Martin, A. Assmann, A. Vegh, A. Memic, B. D. Olsen, N. Annabi, A. Khademhosseini, Advanced Functional Materials 2015, 25, 4814; b) C. Williams, J. Liao, E. M. Joyce, B. Wang, J. B. Leach, M. S. Sacks, J. Y. Wong, Acta Biomaterialia 2009, 5, 993.
[20] M. Ghovvati, S. Baghdasarian, A. Baidya, J. Dhal, N. Annabi, Journal of Biomedical Materials Research Part B: Applied Biomaterials 2022, 110, 1511.
[21] S. Baghdasarian, B. Saleh, A. Baidya, H. Kim, M. Ghovvati, E. S. Sani, R. Haghniaz, S. Madhu, M. Kanelli, I. Noshadi, N. Annabi, Materials Today Bio 2022, 13, 100199.
[22] a) C. Zhang, Y. Zhou, H. Han, H. Zheng, W. Xu, Z. Wang, ACS Nano 2021, 15, 1785; b) P. Zarrintaj, E. Zangene, S. Manouchehri, L. M. Amirabad, N. Baheiraci, M. R. Hadjighasem, M. Farokhi, M. R. Ganjali, B. W. Walker, M. R. Sacb, M. Mozafari, S. Thomas, N. Annabi, Applied Materials Today 2020, 20, 100784; c) J. Park, J. Jeon, B. Kim, M. S. Lee, S. Park, J. Lim, J. Yi, H. Lee, H. S. Yang, J. Y. Lee, Advanced Functional Materials 2020, 30, 2003759; d) Y. Li, L. Wei, L. Lan, Y. Gao, Q. Zhang, H. Dawit, J. Mao, L. Guo, L. Shen, L. Wang, Acta Biomater 2022, 139, 157.
[23] a) S. Liang, Y. Zhang, H. Wang, Z. Xu, J. Chen, R. Bao, B. Tan, Y. Cui, G. Fan, W. Wang, W. Wang, W. Liu, Adv Mater 2018, 30, e1704235; b) S. Kim, L. K. Jang, M. Jang, S. Lee, J. G. Hardy, J. Y. Lee, ACS Applied Materials & Interfaces 2018, 10, 33032; c) S. Houshyar, M. M. Pillai, T. Saha, G. Sathish-Kumar, C. Dekiwadia, S. R. Sarker, R. Sivasubramanian, R. A. Shanks, A. Bhattacharyya, RSC Advances 2020, 10, 40351.
[24] a) S. Zhang, Y. Chen, H. Liu, Z. Wang, H. Ling, C. Wang, J. Ni, B. çelebi-Saltik, X. Wang, X. Meng, H.-J. Kim, A. Baidya, S. Ahadian, N. Ashammakhi, M. R. Dokmeci, J. Travas-Sejdic, A. Khademhosseini, Advanced Materials 2020, 32, 1904752; b) C. Xie, X. Wang, H. He, Y. Ding, X. Lu, Advanced Functional Materials 2020, 30, 1909954; c) D. Ohayon, S. Inal, Advanced Materials 2020, 32, 2001439; d) G. Balakrishnan, J. Song, C. Mou, C. J. Bettinger, Advanced Materials 2022, 34, 2106787.
[25] N. Kostevšek, C. C. L. Cheung, I. Serša, M. E. Kreft, I. Monaco, M. Comes Franchini, J. Vidmar, W. T. Al-Jamal, Nanomaterials (Basel) 2020, 10, 889.
[26] D. Gan, T. Xu, W. Xing. M. Wang. J. Fang, K. Wang, X. Ge, C. W. Chan, F. Ren, H. Tan, X. Lu, Journal of Materials Chemistry B 2019, 7, 1716.
[27] J. R. Soucy, E. Shirzaci Sani, R. Portillo Lara, D. Diaz, F. Dias, A. S. Weiss, A. N. Koppes, R. A. Koppes, N. Annabi, Tissue Engineering Part A 2018, 24, 1393.

1. A composition of matter comprising a hydrogel and a polymer comprising dopamine having a nitro (NO₂) moiety coupled to an aromatic ring (“nDopa”).

2. The composition of claim 1, wherein the nDopa further comprises a methacrylate moiety on the aromatic ring (“nMAD”).

3. The composition of claim 2, wherein methacrylate moieties in the composition are coupled so as to form a polymeric backbone on which nDopa is disposed so as to facilitate chemical suturing of aromatic domains of nDopa molecules.

4. The composition of claim 3, wherein the polymeric backbones form sutured-nitrocatecholic strands of nDopa polymers (“S-nCAT”).

5. The composition of claim 4, wherein the composition comprises a gelatin that forms a gelatin-based, S-nCAT/gelatin hydrogel (“S-nCAGE”).

6. The composition of claim 5, further comprising a pharmaceutical excipient selected from the group consisting of a preservative, a tonicity adjusting agent, a detergent, a viscosity adjusting agent, a sugar and a pH adjusting agent.

7. The composition of claim 5, wherein chemical moieties are disposed on the S-nCAGE such that it exhibits one or more of the following materials properties:
an ultimate strength of at least 50 kPa;
a tensile toughness of at least 1×10⁴ Jm⁻³);
a tensile modulus of at least 40 kPa;
a Young's modulus of at least 100 kPa;
adhesive energies of at least 5 or 10 Jm⁻²;
ionic conductance of at least 1×10⁻³ S/m; and/or
an adhesive strength of at least 10, 20, 30 or 40 kPa.

8. The composition of claim 5, further comprising one or more therapeutic agents selected from an anti-inflammatory agent, an agent that modulates coagulation, an antibiotic agent, or a chemotherapeutic agent.

9. The composition of claim 5, further comprising mammalian cells.

10. A method of making a hydrogel composition comprising sutured-nitrocatecholic dopamine polymers, the method comprising combining together a hydrogel and polymers comprising a polymeric backbone comprising nDopa disposed on the polymeric backbone so as to facilitate chemical suturing of aromatic domains of nDopa molecules; wherein the hydrogel and the polymers are combined so as to form sutured-nitrocatecholic nDopa polymers within the hydrogel composition.

11. The method of claim 10, further comprising disposing in the hydrogel composition:
a pharmaceutical excipient selected from the group consisting of a preservative, a tonicity adjusting agent, a detergent, a viscosity adjusting agent, a sugar and a pH adjusting agent;
one or more therapeutic agents selected from an anti-inflammatory agent, an agent that modulates coagulation, an antibiotic agent, a chemotherapeutic agent; and/or
mammalian cells.

12. The method of claim 11, wherein the hydrogel composition comprises a gelatin.

13. The method of claim 10, wherein the hydrogel and the polymers are formed in an oxidative environment.

14. The method of claim 10, wherein the nDopa comprises a methacrylate moiety coupled to the aromatic ring.

15. The method of claim 10, wherein the hydrogel composition is made to exhibit one or more of the following materials properties:
an ultimate strength of at least 50 kPa;
a tensile toughness of at least 1×10⁴ Jm⁻³);
a tensile modulus of at least 40 kPa;
a Young's modulus of at least 100 kPa;
adhesive energies of at least 5 or 10 Jm⁻²;
ionic conductance of at least 1×10⁻³ S/m; and/or
an adhesive strength of at least 10, 20, 30 or 40 kPa.

16. A method of adhering a first wet tissue to a second wet tissue comprising disposing a S-nCAGE composition of claim 5 between the first wet tissue and the second wet tissue so as to couple the first wet tissue to the second wet tissue, and adhering the first wet tissue and the second wet tissue by using the S-nCAGE as an adhesive composition.

17. The method of claim 16, wherein the first wet tissue and the second wet tissue are adhered in vivo.

18. The method of claim 16, wherein the adhesive composition comprises:
a pharmaceutical excipient selected from the group consisting of a preservative, a tonicity adjusting agent, a detergent, a viscosity adjusting agent, a sugar and a pH adjusting agent;
and/or one or more therapeutic agents such as an anti-inflammatory agent, an agent that modulates coagulation, an antibiotic agent, a chemotherapeutic agent or the like; and/or
mammalian cells.

19. The method of claim 16, wherein the method comprises a step in which moieties in the S-nCAGE are covalently crosslinked.

20. The method of claim 16, wherein chemical moieties are disposed on the S-nCAGE such that it exhibits one or more of the following materials properties:
an ultimate strength of at least 50 kPa;
a tensile toughness of at least 1×10⁴ Jm⁻³);
a tensile modulus of at least 40 kPa;
a Young's modulus of at least 100 kPa;
adhesive energies of at least 5 or 10 Jm⁻²;
ionic conductance of at least 1×10⁻³ S/m; and/or
an adhesive strength of at least 10, 20, 30 or 40 kPa.
